(19) United States
(12) Patent Application Publication
(10) Pub. No.: US 2008/0274011 A1
(43) Pub. Date: Nov. 6, 2008

(54) SHAPE MEMORY ALLOY ENDOPROSTHESIS DELIVERY SYSTEM

(75) Inventors: Joseph Michael FLOMENBLIT, Holon (IL); Nathalie Boris BUDIGINA, Holon (IL)

Correspondence Address: CADWALADER, WICKERSHAM & TAFT LLP, ONE WORLD FINANCIAL CENTER, NEW YORK, NY, 10281-2101, US

(73) Assignee: MEDINOL LTD.

(21) Appl. No.: 12/138,242

(22) Filed: Jun. 12, 2008

Related U.S. Application Data
(62) Division of application No. 10/462,676, filed on Jun. 17, 2003.

(51) Int. Cl. A61L 2/04

(52) U.S. Cl. 422/1

(57) ABSTRACT

In accordance with embodiments of the present invention, a method for preparing a shape memory alloy endoprosthesis, displaying strain induced martensite phenomenon, for delivery includes inserting a shape memory alloy endoprosthesis into a delivery device, inducing a first strain within a first region of the shape memory alloy endoprosthesis, inducing a second strain within a second region of the shape memory alloy endoprosthesis, and sterilizing the delivery device while maintaining the first strain and the second strain induced within the shape memory alloy endoprosthesis. In accordance with other embodiments of the present invention, an apparatus for delivering a shape memory alloy endoprosthesis includes an inner core having a first diameter, an outer body having a second diameter greater than the first diameter, and a calibrated endcap attached to the inner core. The outer body surrounds the inner core, and the calibrated endcap includes a roof section having a third diameter greater than the first diameter and less than the second diameter.

### TECHNICAL FIELD

The present invention relates to a delivery method, apparatus and system for an endoprosthesis. More particularly, the present invention relates to a delivery method, apparatus and system for a shape memory alloy endoprosthesis which displays strain induced martensite phenomenon.

### BACKGROUND OF THE INVENTION

Implantable endoprostheses, such as, for example, stents, heart valves, bone plates, anchors, screws, clips, etc., must meet many requirements to be useful and safe for their intended purpose. For example, they must be chemically and biologically inert to living tissue and to be able to stay in position over extended periods of time. Furthermore, devices of the kind mentioned above must have the ability to expand from a contracted state, which facilitates insertion into body cavities, conduits, lumens, etc., to a useful expanded diameter. This expansion is either accomplished by a forced expansion, such as in the case of certain kinds of stent by the action of a balloon-ended catheter, or by self-expansion such as by shape-memory effects.

A widely used metal alloy for such applications is the nickel-titanium (Ni—Ti) binary alloy generally known as “Nitinol.” Under certain conditions, Nitinols can be highly elastic such that they are able to undergo extensive deformation and yet return to their original shape. Furthermore, Nitinols possess shape memory properties such that they can “remember” a specific shape imposed during a particular heat treatment and can return to that imposed shape under certain conditions. Other shape memory alloys are also known, such as, for example, the Ni—Ti—X ternary alloy (where X may be V, Co, Cu, Fe, etc.), the Cu-AT-Ni ternary alloy, the Cu—Zn-AT ternary alloy, etc.

The shape memory effect demonstrated by Nitinol alloys generally results from metallurgical phase properties. Certain Nitinol alloys are characterized by a transition temperature range, above which the predominant metallurgical phase is termed “austenite,” and below which the predominant metallurgical phase is termed “martensite.” The transformation temperature from martensite to austenite is termed as “austenitic transformation,” while the reverse transformation, from austenite (or austenitic state) to martensite (or martensitic state), is termed “martensitic transformation.” These phase transformations occur over a range of temperatures and are commonly discussed with reference to temperatures A_{S} and A_{F}, the start and finish temperatures of the austenitic transformation, respectively, and with reference to temperatures M_{S} and M_{F}, the start and finish temperatures of the martensitic transformation, respectively. The martensitic transformation temperature range is lower than the austenitic transformation temperature range, with the various temperatures related, generally, as follows: M_{F}<M_{S}<A_{S}<A_{F}.

Transformation between these two phases is reversible such that the alloys may be treated to assume different shapes or configurations in the two phases and can reversibly switch between one shape to another when transformed from one phase to the other. In the case of Nitinol medical devices, it is preferable that they remain in the austenitic state while deployed in the body because Nitinol austenite is stronger and less deformable, and thus more resistant to external forces, than Nitinol martensite. These phase transformations may be induced through changes in temperature, or, alternatively, through changes in stress or strain. For example, a Nitinol medical device may be formed in an austenitic state, and then deformed to such an extent that some or all of the austenite transforms to strain-induced martensite.

A strain-induced martensitic phase transformation may alter the austenitic transformation temperatures of the Nitinol device, typically by increasing the austenitic start and finish temperatures, A_{S} and A_{F}, to within several degrees below, or above, normal body temperature (37° C.). The degree to which A_{S} and A_{F} are increased depends upon the degree of the induced strain. Additionally, different regions of the Nitinol device may be subjected to different strains, resulting in different austenitic transformation start temperatures, such as, for example, A_{S}, and A_{S2}, for Regions 1 and 2, respectively.

In one embodiment, A_{S1}<A_{S2}<T_{body}. In this embodiment, each region may individually begin the austenitic transformation as the Nitinol device reaches the corresponding austenitic transformation start temperature. However, because austenitic transformation start temperatures are different, each region will experience different transformation kinetics, with Region 1 typically experiencing austenitic transformation before Region 2. In another embodiment, A_{S1}<T_{body}<A_{S2}. In this embodiment, Region 1 may complete the austenitic transformation under the influence of body temperature, while Region 2 may require another mechanism to start the austenitic transformation, such as, for example, additional heating, mechanical deformation, etc.

Implantable medical devices made of Nitinol are known in the art. For example, U.S. Pat. No. 5,562,641 to Flomenblit et al. discloses a two-way shape memory alloy stent having an austenitic transformation temperature range that is above body temperature and a martensitic transformation temperature range that is below body temperature. The last conditioned state (i.e., austenite or martensite) of this two-way shape memory alloy stent is thereby retained at body temperature. In another example, U.S. Pat. No. 5,624,508 to Flomenblit et al. discloses a method for manufacturing shape memory alloy devices exhibiting thermally-induced, two-way shape memory effects. In a further example, U.S. Pat. No. 5,876,434 to Flomenblit et al. discloses an implantable shape memory alloy device which is expanded from a strain-induced martensitic state to a stable austenitic state when temperature is above increased A_{S}′>A_{S}°. This shape memory alloy device may, or may not, remain in the deformed martensitic, or partially martensitic, state without the use of a restraining member. Different regions of the stent may be deformed to different strains, resulting in different austenitic transformation temperature ranges, and, consequently, different shape recovery kinetics in those regions.

A strain-induced martensitic stent having different deformation regions may be loaded into a delivery system and then sterilized at temperatures exceeding the different austenitic transformation temperature ranges within the stent. During the sterilization process, however, the different strains induced within the different deformation regions are equalized to a common strain provided by a restraining member of the delivery system, such as, for example, an outer body of a delivery device. Unfortunately, the common strain also provides a common austenitic transformation temperature range, thereby defeating the purpose of inducing multiple deformation regions having different strains, austenitic transformation temperature ranges and shape recovery kinetics.

Devices for implanting self-expanding stents are likewise known in the art. For example, U.S. Pat. No. 5,484,444 to Braunschweiler et al. discloses a device for implanting a radially self-expanding stent that includes an outer body and an inner core element having a stamped region which complements the surface of the stent and facilitates implantation. The self-expanding stent is compressed, or folded, onto the inner core and expands immediately into the inner diameter of the body cavity, vessel, etc., as the outer body is pulled back over the inner core. Unfortunately, the sharp, leading edge of the stent may damage the internal surface of the vessel as the stent is released and immediately begins to expand. Moreover, as discussed in Braunschweiler, once the stent is partially released, it can only be pulled proximally and not pushed distally, because if the stent were to be pushed, the expanded, distal end would inevitably injure the vessel in which it was introduced.

### SUMMARY OF THE INVENTION

In accordance with embodiments of the present invention, a method for preparing a shape memory alloy endoprosthesis, displaying strain induced martensite phenomenon, for delivery includes inserting a shape memory alloy endoprosthesis into a delivery device, inducing a first strain within a first region of the shape memory alloy endoprosthesis, inducing a second strain within a second region of the shape memory alloy endoprosthesis, and sterilizing the delivery device while maintaining the first strain and the second strain induced within the shape memory alloy endoprosthesis.

In accordance with other embodiments of the present invention, an apparatus for delivering a shape memory alloy endoprosthesis includes an inner core having a first diameter, an outer body having a second diameter greater than the first diameter, and a calibrated endcap attached to the inner core. The outer body surrounds the inner core, and the calibrated endcap includes a roof section having a third diameter greater than the first diameter and less than the second diameter.

### BRIEF DESCRIPTION OF THE DRAWINGS

FIG. 1 is a schematic representation of a delivery system for a shape memory alloy endoprosthesis, according to an embodiment of the present invention.

FIG. 2 is a schematic representation of a delivery system depicting a partially-deployed shape memory alloy endoprosthesis, according to an embodiment of the present invention.

FIG. 3 is a flow chart depicting a method for preparing a shape memory alloy medical endoprosthesis for delivery, according to an embodiment of the present invention.

### DETAILED DESCRIPTION

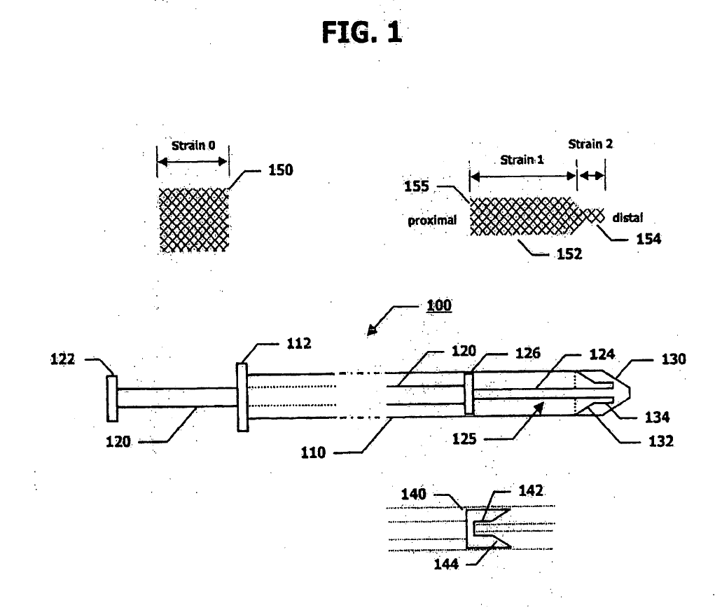

FIG. 1 is a schematic representation of a delivery system for a shape memory alloy endoprosthesis, according to an embodiment of the present invention.

Referring to FIG. 1, delivery system 100 generally includes flexible outer body 110, flexible inner core 120 and calibrated endcap 130. In an embodiment, outer body 110 and inner core 120 may be generally circular in cross-section, while calibrated endcap 130 may be circular, conical, etc., in cross-section. Calibrated endcap 130 may be fixedly attached to inner core 120 (e.g., adhesive, etc.), or, alternatively, calibrated endcap 130 may be removably attached to inner core 120 (e.g., screw/thread, etc.), thereby facilitating the use of different types of removable calibrated endcaps 130 within delivery system 100. In an embodiment, inner core 120 and calibrated endcap 130 may include an interior cavity, or lumen, in which a guide wire, fiber optic lens/cable assembly, etc., may be inserted (not shown for clarity).

In an embodiment, inner core 120 may be longer than outer body 110, and delivery system 100 may include outer handle 112, attached to the proximal end of outer body 110, and inner handle 122, attached to the proximal end of inner core 120. In this embodiment, outer handle 110 and inner handle 120 may provide convenient surfaces upon which to apply the appropriate forces necessary to slide outer body 110 over inner core 120, in the proximal direction, during the deployment of the shape memory alloy endoprosthesis.

Inner core 120 may include shoulder 126, located near the distal end of inner core 120. In an embodiment, shoulder 126 may be circular in cross-section. In this embodiment, the diameter of shoulder 126 may be slightly less than the diameter of outer body 110 in order to prevent lateral motion of the shape memory alloy endoprosthesis in the proximal direction during deployment, while at the same time permitting relative motion between outer body 110 and inner core 120. In another embodiment, a gasket may be attached to the outer surface of shoulder 126 to prevent proximally-directed fluid flow, either before, during or after deployment. Additionally, the gasket may reduce the nominal coefficient of friction between outer body 110 and shoulder 126, thereby improving the relative motion between outer body 110 and inner core 120. In one embodiment, shoulder 126 may include x-ray opaque material, while in another embodiment, shoulder 126 may include radio-frequency opaque material. Generally, shoulder 126 may optionally include one or more materials capable of reflecting medical imaging device emissions to facilitate location of the distal end of delivery system 100 within the body.

Inner core 120 may include forward section 124, located at the distal end of inner core 120 and extending from shoulder 126 to endcap 130. In one embodiment, the diameter of forward section 124 may be less than the diameter of inner core 120 proximal to shoulder 126, while in another embodiment, the diameter of forward section 124 may be equal to, or greater than, the diameter of inner core 120 proximal to shoulder 126. The diameter of forward section 124 may be constant along its length, or, alternatively, the diameter of forward section 124 may vary along its length. A shape memory alloy endoprosthesis may be fitted within payload volume 125, generally defined by outer body 110, shoulder 126, forward section 124 and calibrated endcap 130.

Calibrated endcap 130 may include transition section 132 and roof section 134, and may optionally include one or more materials capable of reflecting medical imaging device emissions to facilitate location of the distal end of delivery system 100 within the body. In an embodiment, transition section 132 may provide a reduction in diameter, generally, from the diameter of outer body 110 to the diameter of roof section 134. As depicted in FIG. 1, the diameter of roof section 134 may be less than the diameter of outer body 110 but more than the diameter of forward section 124. The distal portion of a shape memory alloy endoprosthesis may be captured by calibrated endcap 130 and deformed to a diameter smaller than the remaining, proximal portion of the shape memory alloy endoprosthesis housed within payload volume 125 and generally restrained by outer body 110. Importantly, the reduction in diameter of the distal portion of the shape memory alloy endoprosthesis imparts an increase in strain compared to the remaining, proximal portion of the shape memory alloy endoprosthesis. Advantageously, the dimensions of calibrated endcap 130, such as, for example, the diameter of roof section 134, the length of roof section 134, the length of transition section 132, etc., may correlate to a specific increase in strain for a particular shape memory alloy endoprosthesis.

An exemplary shape memory alloy endoprosthesis is also depicted in FIG. 1, both in a deployed configuration (stent 150) and in an undeployed configuration (stent 155). In an embodiment, the shape memory alloy endoprosthesis may be constructed of Nitinol and may include residual strain e0 (ε₀) when deployed in an austenitic state, generally corresponding to stent 150. In this embodiment, the diameter of stent 150 may be greater than the diameter of outer body 110. When inserted within delivery system 100, however, a different configuration, generally corresponding to stent 155, may be assumed. In this configuration, some portion of stent 155 may be deformed to a particular strain e1 (ε₁) by outer body 110, such as, for example, body 152, while a smaller portion of stent 155 may be deformed to a particular strain e2 (ε₂) by calibrated endcap 130, such as, for example, leading edge 154. In an embodiment, the proximal portion of leading edge 154 may be deformed to a particular strain profile by transition section 132, while the distal portion of leading edge 154 may be deformed to a constant strain by roof section 134. In other words, leading section 154 may include a smaller, proximal portion, in which the strain varies from e1 (ε₁) to e2 (ε₂) according to a particular profile (e.g., linear, parabolic, etc.), and a larger, distal portion, in which the strain is essentially constant at e2 (ε₂).

After deformation by delivery system 100, stent 155 may contain regions in which the austenite transformation temperatures differ from one another, such as, for example, body 152 and leading edge 154. In an embodiment, body 152 may experience strain e1 (ε₁) producing austenitic transformation temperatures A_{S1} and A_{F1}, while the larger, distal portion of leading edge 154 may generally experience strain e2 (ε₂) producing austenitic transformation temperatures A_{S2} and A_{F2}. For simplicity, the effects of the strain profile experienced by the smaller, proximal portion of leading edge 154 may be neglected. In one embodiment, e2 (ε₂) may be greater than e1 (ε₁), and all of the austenitic transformation temperatures may be below body temperature, i.e., A_{S1}<A_{S3}, A_{F1}<A_{F3}, and A_{S1}, A_{S2}, A_{F1}, A_{F3}<T_{body}. In another embodiment, e2 (ε₂) may be greater than e1 (ε₁), and only the austenitic transformation temperatures associated with the e1 (ε₁) region may be below body temperature, i.e., A_{S1}<A_{S3}, A_{F1}<A_{F3}, and A_{S1}, A_{F1}<T_{body}<A_{S3}, A_{F3}. In this embodiment, an alternative mechanism may be required to deploy the e2 (ε₂) region after initial deployment, such as, for example, additional heating using a warm saline solution, mechanical deformation using a balloon catheter, etc.

In an alternative embodiment, calibrated shoulder 140 may replace shoulder 126, and may include a calibrated section similar In design and function to the elements of calibrated endcap 130. For example, calibrated shoulder 140 may include transition section 142 and roof section 144. Transition section 142 may provide a reduction in diameter, generally, from the diameter of outer body 110 to the diameter of roof section 144, which may be less than the diameter of outer body 110 but more than the diameter of forward section 124. In this manner, the proximal portion of a shape memory alloy endoprosthesis may be captured by calibrated shoulder 140 and deformed to a diameter smaller than the remaining, distal portion of the shape memory alloy endoprosthesis housed within payload volume 125. Importantly, the reduction in diameter of the proximal portion of the shape memory alloy endoprosthesis imparts an Increase in strain compared to the remaining portion of the shape memory alloy endoprosthesis. Delivery system 100 may include either calibrated endcap 130 or calibrated shoulder 140, or, alternatively, both calibrated endcap 130 and calibrated shoulder 140.

Advantageously, the dimensions of calibrated shoulder 140, such as, for example, the diameter of roof section 144, the length of roof section 144, the length of transition section 142, etc., may correlate to a specific increase in strain for a particular shape memory alloy endoprosthesis. In an embodiment, the strain induced by calibrated shoulder 140, e3 (ε₃), may be greater than e1 (ε₁), and all of the austenitic transformation temperatures may be below body temperature, i.e., A_{S1}<A_{S3}, A_{F1}<A_{F3}, and A_{S1}, A_{S3}, A_{F1}, A_{F}3<T_{body}. In another embodiment, e3 (ε₃) may be greater than e1 (ε₁), and only the austenitic transformation temperatures associated with the e1 (ε₁) region are below body temperature, i.e., A_{S1}<A_{S3}, A_{F1}<A_{F3}, and A_{S1}, A_{F1}<T_{body}<A_{S3}, A_{F3}. In this embodiment, an alternative mechanism may be required to deploy the e3 (ε₃) region after deployment, such as, for example, additional heating using a warm saline solution, mechanical deformation using a balloon catheter, etc.

In a further embodiment, delivery system 100 may include cooling fluid to maintain the temperature of the shape memory alloy endoprosthesis below the various austenitic transformation finish temperature until deployment. For example, cooling fluid may be introduced into an inner lumen, extending through the entire length of inner core 120 to payload volume 125, and may be returned through an outer lumen defined by outer body 110 and inner core 120 proximal to shoulder 126. In this embodiment, forward section 124 may include one or more holes through which the cooling fluid may flow into payload volume 125, and shoulder 126 may include one or more holes, cutouts, etc., to facilitate fluid flow from payload volume 125 to the outer lumen. In this manner, the shape memory alloy endoprosthesis captured within payload volume 125 may be maintained at an appropriate temperature in order to prevent instantaneous austenitic phase transformation, caused by heat transfer during advancement of delivery system 100 within the body, upon deployment.

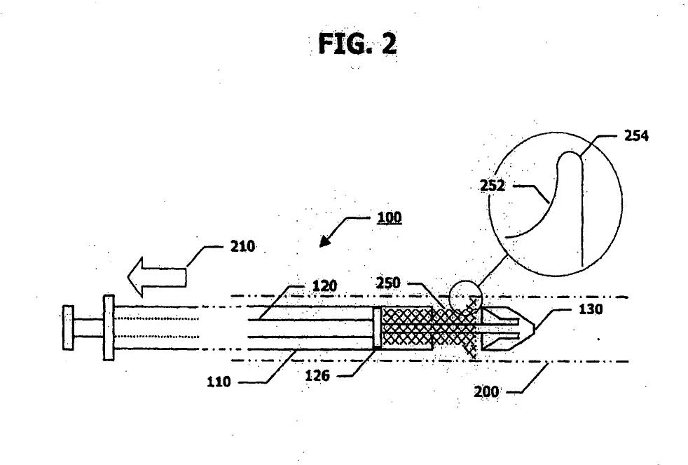

FIG. 2 is a schematic representation of a delivery system for a shape memory alloy endoprosthesis, depicted in a partially deployed state, according to an embodiment of the present invention.

Referring to FIG. 2, delivery system 100 is depicted in a partially deployed state, in which stent 250 may be in transition from a loaded configuration within delivery system 100 to a deployed configuration within body lumen 200. In an embodiment, stent 250 may include at least two regions of induced strain, each having a different austenitic transformation temperature range. During the deployment process, heat flow from body lumen 200 increases the temperature of stent 250. Austenitic phase transformation may occur within each region of induced strain as the temperature of stent 250 passes through each specific austenitic transformation temperature range. Because each region of induced strain may have a different austenitic transformation temperature range, and because a temperature gradient may be established over the length of stent 250 during the deployment process, austenitic transformation may occur at different times for different regions of stent 250.

For example, stent 250 may include a region of induced strain e1 (ε₁), such as body 252, and a region of induced strain e2 (ε₂), such as leading edge 254. In this example, e1 (ε₁) may be less than e2 (ε₂), and the austenitic transformation temperature range associated with body 252 may be less than the austenitic transformation temperature range associated with leading edge 254. Accordingly, as stent 250 begins to deploy, heat flow from body lumen 200 may increase the temperature of stent 250 such that body 252 begins austenitic transformation before leading edge 254. The austenitic transformation lag experienced by leading edge 254 effectively blunts the sharp edge of the expanding distal portion of stent 250, thereby preventing damage to the walls of body lumen 200 which may occur during the initial deployment stages of a typical shape memory alloy endoprosthesis. Additionally, partially-deployed stent 250 may be repositioned within body lumen 200, in both the proximal and distal directions, without damaging the walls of body lumen 200.

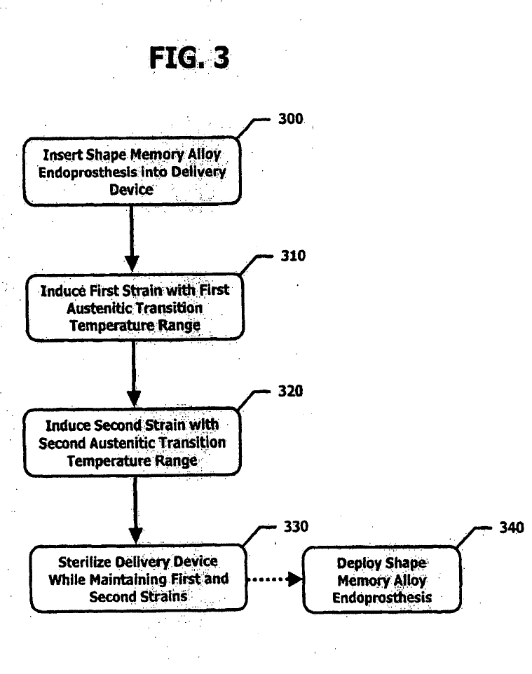

FIG. 3 is a flow chart depicting a method for preparing a shape memory alloy endoprosthesis for delivery, according to an embodiment of the present invention.

In an embodiment, a shape memory alloy endoprosthesis may be inserted (300) into a delivery device. In an embodiment, inner core 120 may be fixed and outer body 110 may be advanced in the proximal direction so that the distal end of outer body 110 approaches shoulder 126, thereby exposing at least a portion of forward section 124. In another embodiment, outer body 110 may be fixed and inner core 120 may be advanced in the distal direction so that shoulder 126 approaches the distal end of outer core 110, thereby exposing at least a portion of forward section 124. Calibrated endcap 130 may be passed through the center of stent 150, and stent 150 may then be generally aligned over forward section 124.

In one embodiment, stent 150 may be deformed to a smaller diameter and then inserted (300) into delivery system 100. The distal portion of stent 150 may be inserted into calibrated endcap 130 and advanced to roof section 134. The proximal portion of stent 150 may be inserted, generally, towards shoulder 126 and then the distal portion of delivery system 100 may be closed, for example, by fixing outer body 110 and advancing inner core 120 in proximal direction, by fixing inner core 120 and advancing outer body 110 in a distal direction, etc. As noted above, stent 155 represents the undeployed, or loaded, configuration of stent 150. In an alternative embodiment, the proximal portion of stent 150 may be inserted into calibrated shoulder 140 and advanced to roof section 144.

A first strain, having a first austenitic transition temperature range, may be induced (310) within a first region of the shape memory alloy endoprosthesis. In an embodiment, outer body 110 of delivery system 100 may induce a particular strain e1 (ε₁) within a proximal portion of stent 155, such as, for example, body 152. This strain may produce an austenitic transformation temperature range generally denoted by start and finish temperatures, A_{S1} and A_{F1}, respectively. In one embodiment, this austenitic transformation temperature range may be below normal body temperature.

A second strain, having a second austenitic transition temperature range, may be induced (320) within a second region of the shape memory alloy endoprosthesis. In an embodiment, roof section 134 of delivery system 100 may induce (320) a particular strain e2 (ε₂), greater than e1 (ε₁), within a distal portion of stent 155, such as, for example, leading edge 154. This strain may produce an austenitic transformation temperature range generally denoted by start and finish temperatures, A_{S2} and A_{F2}, respectively. In one embodiment, this austenitic transformation temperature range may be below normal body temperature, while in another embodiment, this austenitic transformation temperature range may be above normal body temperature.

In an alternative embodiment, roof section 144 of delivery system 100 may induce (320) a particular strain e3 (ε₃) within a proximal portion of stent 155, such as, for example, the trailing edge of body 152. This strain may produce an austenitic transformation temperature range generally denoted by start and finish temperatures, A_{S3} and A_{F3}, respectively.

The delivery device may be sterilized (330) at a temperature above the first austenitic transition temperature range and second austenitic transition temperature range while maintaining the first strain and the second strain. In an embodiment, delivery system 100, containing stent 155, may be sterilized (330) at a temperature above the austenitic transformation temperature ranges associated with the various regions of induced strain, such as, for example, e1 (ε₁), e2 (ε₂), etc. Due to the constraining effects of delivery system 100, and, in particular, outer body 110 and calibrated endcap 130, stent 155 may not undergo strain equalization normally experienced during high-temperature sterilization. Rather, after the sterilization process concludes, the various regions of induced strain within stent 155, such as, for example, e1 (ε₁), e2 (ε₂), etc., may be preserved by delivery system 100. Importantly, the austenitic transformation temperature ranges associated with each region of induced strain will also be preserved. Accordingly, each region of induced strain may experience different kinetics upon deployment within the body. For sterilization processes occurring below these austenitic transformation temperature ranges, delivery system 100 also preserves the various regions of induced strain within stent 155.

In a further embodiment, the shape memory alloy endoprosthesis may be deployed (340) from the delivery device. Generally, delivery system 100 may be introduced into a body lumen, cavity, etc., and advanced to the deployment location. In an embodiment, inner core 120 of delivery system 100 may be fixed during deployment while outer body 110 may be advanced in a proximal direction, as indicated, generally, by directional arrow 210. This relative motion between inner core 120 and outer body 110 gradually exposes stent 250 to body lumen 200, as well as to any fluid which may be present therein. Heat flow between body lumen 200 and stent 250 may depend, generally, upon various factors, including, for example, the temperature different between body lumen 200 and stent 250, the heat conductivity coefficient α, etc. As the temperature of stent 250 increases due to this heat flow, austenitic phase transformation may occur and stent 250 may then assume the deployed configuration within body lumen 200.

Several embodiments of the present invention are specifically illustrated and described herein. However, it will be appreciated that modifications and variations of the present invention are covered by the above teachings and within the purview of the appended claims without departing from the spirit and intended scope of the invention.

1. A method for preparing a shape memory alloy endoprosthesis, displaying strain induced martensite phenomenon, for delivery, comprising:
inserting a shape memory alloy endoprosthesis, having an austenitic state and a martensitic state, into a delivery device;
inducing a first strain, having a first austenitic transition temperature range, within a first region of the shape memory alloy endoprosthesis;
inducing a second strain, having a second austenitic transition temperature range, within a second region of the shape memory alloy endoprosthesis; and
sterilizing the delivery device at a temperature above the first austenitic transition temperature range and second austenitic transition temperature range while maintaining the first strain and the second strain.

2. The method of claim 1, further comprising deploying the shape memory alloy endoprosthesis from the delivery device.

3. The method of claim 2, wherein the shape memory alloy endoprosthesis is a Nitinol stent.

4. The method of claim 3, wherein first strain is less than the second strain.

5. The method of claim 4, wherein the first austenitic transition temperature range is less than the second austenitic transition temperature range.

6. The method of claim 5, wherein the first austenitic transition temperature range is less than normal body temperature.

7. The method of claim 6, wherein the second austenitic transition temperature range is greater than normal body temperature.

8. The method of claim 3, wherein the first strain is induced by reducing a first diameter of the first portion of the Nitinol stent.

9. The method of claim 8 wherein the second strain is induced by reducing a second diameter of the second portion of the Nitinol stent, the second diameter being less than the first diameter.

10. The method of claim 3, wherein said deploying the shape memory alloy endoprosthesis includes fixing an inner core of the delivery device in place and pulling back an outer body of the delivery device to expose the shape memory alloy endoprosthesis.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)
